(19) United States
(12) Patent Application Publication
(10) Pub. No.: US 2026/0240518 A1
(43) Pub. Date: Aug. 20, 2026

(54) TRANSDUCER ASSEMBLIES FOR INTRAVASCULAR ULTRASOUND PROBES AND RELATED SYSTEMS AND METHODS

(71) Applicant: BARD PERIPHERAL VASCULAR, INC., Franklin Lakes, NJ (US)

(72) Inventors: Peng Guo, Shanghai (CN); Feipeng Guo, Shanghai (CN); Hely Li, Shanghai (CN)

(21) Appl. No.: 19/053,848

(22) Filed: Feb. 14, 2025

(51) Int. Cl. A61B 8/12; A61B 8/00

(52) U.S. Cl. CPC A61B 8/12; A61B 8/445; A61B 2562/166

(57) ABSTRACT

Embodiments disclosed herein are intravascular ultrasound probes and related systems and methods of manufacture and use. In an embodiment, a transducer assembly for an intravascular ultrasound system includes a piezoelectric element, a substrate, and a wire electrically connected to the piezoelectric element and the substrate. The substrate includes a micro printed circuit board (PCB) having a control circuit. The control circuit is configured to electrically couple to a cable of a catheter of the intravascular ultrasound system. The wire is configured to transmit one or more signals between the control circuit and the piezoelectric element.

### BACKGROUND

Intravascular ultrasound (“IVUS”) is an intravascular imaging modality that can be used in various interventional disciplines to characterize lesion morphology, quantify plaque load, guide device sizing, evaluate device placement, and identify complications. IVUS provides cross-sectional images of vascular structures. IVUS can play an important role in decision making during percutaneous coronary intervention (“PCI”) procedures, including pre-procedure evaluation and post-procedure optimization. IVUS is used to assess plaque nature and characteristics, develop appropriate pretreatment strategies, measure lesion length and reference segment vessel diameter, and select appropriate stent diameter, length, and footing points. Post-PCI, IVUS is used to identify stent expansion and apposition and complications such as stent edge entrapment in order to target and optimize the immediate post-PCI outcomes.

### SUMMARY

Embodiments disclosed herein are transducer assemblies for IVUS probes, and related systems and methods. In an embodiment, a transducer assembly for an intravascular ultrasound system includes a piezoelectric element, a substrate, and a wire electrically connected to the piezoelectric element and the substrate. The substrate includes a printed circuit board (“PCB”) having a control circuit. The control circuit is configured to electrically couple to a cable of a catheter of the intravascular ultrasound system. The wire is configured to transmit one or more signals between the control circuit and the piezoelectric element.

In an embodiment, an imaging catheter for an intravascular ultrasound system includes a shaft having a distal end region, a housing secured to the distal end region of the shaft, a cable extending through the shaft and configured to operably couple to a control system of the intravascular ultrasound system, and a transducer assembly secured to the housing. The transducer assembly includes a piezoelectric element, a substrate, and a wire electrically connected to the piezoelectric element and the substrate. The substrate includes a PCB having a control circuit electrically coupled to the cable. The wire is configured to transmit one or more signals between the control circuit and the piezoelectric element.

In an embodiment, an intravascular ultrasound system includes a control system and an imaging catheter operably coupled to the control system. The imaging catheter includes a shaft having a distal end region, a housing secured to the distal end region of the shaft, a cable extending through the shaft and operably coupled to the control system, and a transducer assembly secured to the housing. The transducer assembly includes a piezoelectric element, a substrate including a PCB having a control circuit electrically coupled to the cable, and a wire electrically connected to the piezoelectric element and the substrate. The wire is configured to transmit one or more signals between the control circuit and the piezoelectric element.

In an embodiment, a method of assembling a transducer is disclosed. The method includes providing a piezoelectric element and a substrate including a PCB having a control circuit. The control circuit is configured to electrically couple to a cable of a catheter of the intravascular ultrasound system. The method also includes wire bonding a wire to the piezoelectric element and the substrate to electrically connect the piezoelectric element and the substrate. The wire is configured to transmit one or more signals between the control circuit and the piezoelectric element.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of an IVUS system, according to an embodiment.

FIG. 2 is a side view of a portion of an imaging catheter of the IVUS system of FIG. 1, according to an embodiment.

FIG. 3 is a circuit diagram of a transducer assembly of an IVUS system, according to an embodiment.

FIG. 4 is a schematic diagram of a transducer assembly of an IVUS system, according to an embodiment.

FIG. 5 is a circuit diagram of a substrate of a transducer assembly of an IVUS system, according to an embodiment.

FIG. 6 is a flow diagram of a method of manufacturing a transducer assembly, according to an embodiment.

### DETAILED DESCRIPTION

Embodiments disclosed herein are transducer assemblies for IVUS use (e.g., probes), and related systems and methods of use and manufacture. In many embodiments, the transducer assemblies disclosed herein result in the technical effect of more efficient assembling of a transducer and/or impedance matching for improved performance of the IVUS probe and system.

Transducer assemblies disclosed herein may include a new substrate in comparison to conventional transducer assemblies, and may use wire bonding and/or an inverted packaging process to achieve the assembling of the substrate and piezoelectric element. By using the wire bonding process, the solder joints on the piezoelectric element can be greatly reduced compared with the assembly of conventional transducer assemblies. Moreover, the reduction of solder joints can improve the effective area of the piezoelectric element, which directly affects the transmitting and receiving performance of the piezoelectric element or wafer. The process can achieve higher production consistency and production efficiency.

In many embodiments, an impedance matching network and bridging circuit (e.g., a control circuit) may be added to the substrate of the transducer assembly to improve the electrical energy transmission efficiency. The impedance matching and bridging circuit may include a first capacitor, a second capacitor, and a resistor positioned between the first capacitor and the second capacitor. The added impedance matching network and bridging circuit (e.g., impedance matching circuit or control circuit) can achieve better transducer transmitting and receiving performance relative to conventional transducer assemblies.

Conventional ultrasonic transducers may include a soldered wire for cable assembling, resulting in soldering joints that are large, a high complexity for processing and assembly, and higher costs for processing and assembly. In conventional ultrasonic transducers, the coaxial cable may be directly soldered to both sides of the ultrasonic transducer, resulting problematic larger welding points, poor consistency of the welding process, and poor ultrasonic electro-acoustic energy conversion efficiency. Conventional ultrasonic transducers also may include a drive signal directly welded to the piezoelectric ceramic through a impedance 50-ohm cable and also lack an impedance matching design, resulting in a poor electrical signal in both transmitting and receiving energy utilization.

In at least one, some, or all embodiments of transducer assemblies disclosed herein, the transducer assembly includes an innovative front-end design of the probe that ultimately achieves improved imaging depth and accuracy of intravascular ultrasound for better diagnostic and therapeutic results. This front end design may include the addition of a substrate and the impedance matching circuit thereon, as well as the use of an improved wire bonding process instead of the traditional soldering process, according to an embodiment.

IVUS systems disclosed herein may be used in various interventional disciplines to characterize lesion morphology, quantify plaque load, guide device sizing, evaluate device placement, and identify complications. IVUS systems disclosed herein are configured to provide cross-sectional images of vascular structures. In many embodiments, IVUS systems disclosed herein may play an important role in decision making during percutaneous coronary intervention (“PCI”) procedures, including pre-procedure evaluation and post-procedure optimization. In many embodiments, IVUS systems disclosed herein are configured for use in peripheral vascular diagnosis. IVUS systems disclosed herein may be used to assess plaque nature and characteristics, develop appropriate pretreatment strategies, measure lesion length and reference segment vessel diameter, and select appropriate stent diameter, length, and footing points. Post-PCI, IVUS systems disclosed herein may be used to identify stent expansion and apposition and complications such as stent edge entrapment in order to target and optimize the immediate post-PCI outcomes.

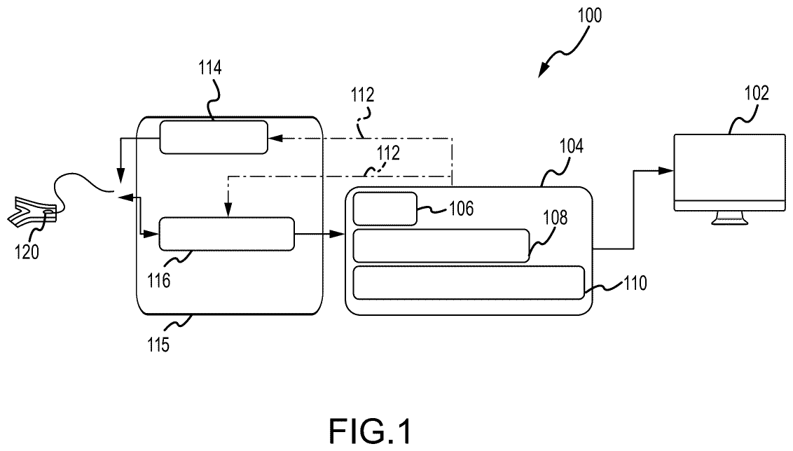

Turning now to the drawings, FIG. 1 is a block diagram of an IVUS system 100, according to an embodiment. The IVUS system may include a control system 104 (e.g., console or integrated power center (“IPC”)), an automatic pullback device 115 operably coupled to the control system 104, and an imaging catheter 120 operably coupled to the pullback device 115. The control system 104 may be part of an electronic device or computer 102 and/or may be operably coupled thereto. The control system 104 may include one or more processors or modules, such as a data acquisition module 106, a signal processing module 108, and an imaging reconstruction module 110. The control system 104 may be electrically or otherwise operably coupled 112 to the automatic pullback device 115. The automatic pullback device may include one or more motors 114 and one or more circuits 116. The one or more circuits may include, for example, a transmitter circuit and/or a receiver circuit operably coupled to the imaging catheter 120 and the control system 104. The pullback device 115 may be configured to pull back the imaging catheter 120 (e.g., the transducer of the imaging catheter 120) along a length of the vessel to allow the imaging catheter 120 to emit and collect ultrasonic signals to form multiple image slices of the vascular cross section that may be integrated to generate a three-dimensional image.

In order to produce the desired image, the imaging catheter 120 may include a transducer at a distal end region thereof that receives one or more electrical signals from the control system 104, and the electrical stimulation of the one or more electrical signals causes the piezoelectric element in the transducer to strain (e.g., expand and contract) to produce high-frequency ultrasound waves. These high-frequency ultrasound waves may be scattered and reflected at the tissue interface, and some of the reflected ultrasound waves are converted back to electrical signals by the transducer. These electrical signals from the reflected ultrasound waves may be analyzed and processed by the imaging reconstruction module 110 in the control system 104 for conversion into grayscale cross-sectional images.

The control system 104 may be used to carry out any of the example methods disclosed herein, such as using a controller. The control system 104 may include at least one computing device. The at least one computing device of the control system 104 may be configured to perform one or more of the acts described herein. The at least one computing device of the control system 104 can include one or more servers, one or more computers (e.g., desk-top computer, lap-top computer), or one or more mobile computing devices (e.g., smartphone, tablet, etc.). In some embodiments, the computing device of the control system 104 comprises the computer 102. The computing device of the control system 104 may include one or more (e.g., all) of the data acquisition module 106, the signal processing module 108, and/or the imaging reconstruction module 110. The computing device of the control system 104 can comprise at least one processor, memory, a storage device, an input/output (“I/O”) device/interface, and a communication interface. In some examples, the at least one computing device of the control system 104 may include a plurality of computing devices, such as a server farm, computational network, or cluster of computing devices.

In some examples, the computing device of the control system 104, the data acquisition module 106, the signal processing module 108, and/or the imaging reconstruction module 110 may include processor(s) having hardware for executing instructions (e.g., instructions for carrying out one or more portions of any of the methods disclosed herein), such as those making up a computer program. For example, to execute instructions, the processor(s) may retrieve (or fetch) the instructions from an internal register, an internal cache, the memory, or a storage device and decode and execute them. In particular examples, processor(s) may include one or more internal caches for data such. As an example, the processor(s) may include one or more instruction caches, one or more data caches, and one or more translation lookaside buffers (“TLBs”). Instructions in the instruction caches may be copies of instructions in memory or storage device. In some examples, the processor may be configured (e.g., include programming stored thereon or executed thereby) to carry out one or more portions of any of the example methods disclosed herein.

In some examples, the processor of the control system 104 is configured to perform any of the acts disclosed herein or cause one or more portions of the computing device or control system 104 to perform at least one of the acts disclosed herein. Such configuration can include one or more operational programs (e.g., computer program products) that are executable by the at least one processor.

The at least one computing device (e.g., a server) of the control system 104 may include at least one memory storage medium (e.g., memory and/or storage device). The computing device of the control system 104 may include memory, which is operably coupled to the processor(s). The memory may be used for storing data, metadata, and programs for execution by the processor(s). The memory may include one or more of volatile and non-volatile memories, such as Random Access Memory (“RAM”), Read Only Memory (“ROM”), a solid state disk (“SSD”), Flash, Phase Change Memory (“PCM”), or other types of data storage. The memory may be internal or distributed memory.

The computing device of the control system 104 may include the storage device having storage for storing data or instructions. The storage device may be operably coupled to the at least one processor. In some examples, the storage device can comprise a non-transitory memory storage medium, such as any of those described above. The storage device (e.g., non-transitory storage medium) may include a hard disk drive (“HDD”), a floppy disk drive, flash memory, an optical disc, a magneto-optical disc, magnetic tape, or a Universal Serial Bus (“USB”) drive or a combination of two or more of these. The storage device may include removable or non-removable (or fixed) media. The storage device may be internal or external to the computing device. In some examples, the storage device may include non-volatile, solid-state memory. In some examples, the storage device may include ROM. Where appropriate, this ROM may be mask programmed ROM, programmable ROM (“PROM”), erasable PROM (“EPROM”), electrically erasable PROM (“EEPROM”), electrically alterable ROM (“EAROM”), or flash memory or a combination of two or more of these. In some examples, one or more portions of the memory and/or storage device (e.g., memory storage medium(s)) may store one or more databases thereon.

In some examples of the control system 104, data may be stored in a memory storage medium such as one or more of the at least one processor (e.g., internal cache of the processor), memory, or the storage device. In some examples, the at least one processor may be configured to access (e.g., via a bus) the memory storage medium(s) such as one or more of the memory or the storage device. For example, the at least one processor may receive and store the data (e.g., look-up tables) as a plurality of data points in the memory storage medium(s). The at least one processor may execute programming stored therein adapted access the data in the memory storage medium(s). For example, the at least one processor may access data in the memory storage medium(s) such as memory or storage device.

The computing device of the control system 104 also may include one or more I/O devices/interfaces, which are provided to allow a user to provide input to, receive output from, and otherwise transfer data to and from the computing device. These I/O devices/interfaces may include a mouse, keypad or a keyboard, a touch screen, camera, optical scanner, network interface, web-based access, modem, a port, other known I/O devices or a combination of such I/O devices/interfaces. The touch screen may be activated with a stylus or a finger. The I/O devices/interfaces may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces are configured to provide graphical data to a display for presentation to a user. The graphical data may be representative of one or more graphical user interfaces and/or any other graphical content as may serve a particular implementation.

The computing device of the control system 104 can further include a communication interface. The communication interface can include hardware, software, or both. The communication interface can provide one or more interfaces for communication (such as, for example, packet-based communication) between the computing device and one or more additional computing devices or one or more networks. For example, communication interface may include a network interface controller (“NIC”) or network adapter for communicating with an Ethernet or other wire-based network or a wireless NIC (“WNIC”) or wireless adapter for communicating with a wireless network, such as a WI-FI.

Any suitable network and any suitable communication interface may be used. For example, the computing device of the control system 104 may communicate with an ad hoc network, a personal area network (“PAN”), a local area network (“LAN”), a wide area network (“WAN”), a metropolitan area network (“MAN”), or one or more portions of the Internet or a combination of two or more of these. One or more portions of one or more of these networks may be wired or wireless. As an example, one or more portions of the control system 104 or the computing device of the control system 104 may communicate with a wireless PAN (“WPAN”) (such as, for example, a BLUETOOTH WPAN), a WI-FI network, a WI-MAX network, a cellular telephone network (such as, for example, a Global System for Mobile Communications (“GSM”) network), or other suitable wireless network or a combination thereof. The computing device of the control system 104 may include any suitable communication interface for any of these networks, where appropriate.

The computing device of the control system 104 may include a bus. The bus can include hardware, software, or both that couples components of the computing device of the control system 104 to each other. For example, the bus of the control system 104 may include an Accelerated Graphics Port (“AGP”) or other graphics bus, an Enhanced Industry Standard Architecture (“EISA”) bus, a front-side bus (“FSB”), a HYPERTRANSPORT (“HT”) interconnect, an Industry Standard Architecture (“ISA”) bus, an INFINIBAND interconnect, a low-pin-count (LPC) bus, a memory bus, a Micro Channel Architecture (MCA) bus, a Peripheral Component Interconnect (“PCI”) bus, a PCI-Express (“PCIe”) bus, a serial advanced technology attachment (“SATA”) bus, a Video Electronics Standards Association local (“VLB”) bus, or another suitable bus or a combination thereof.

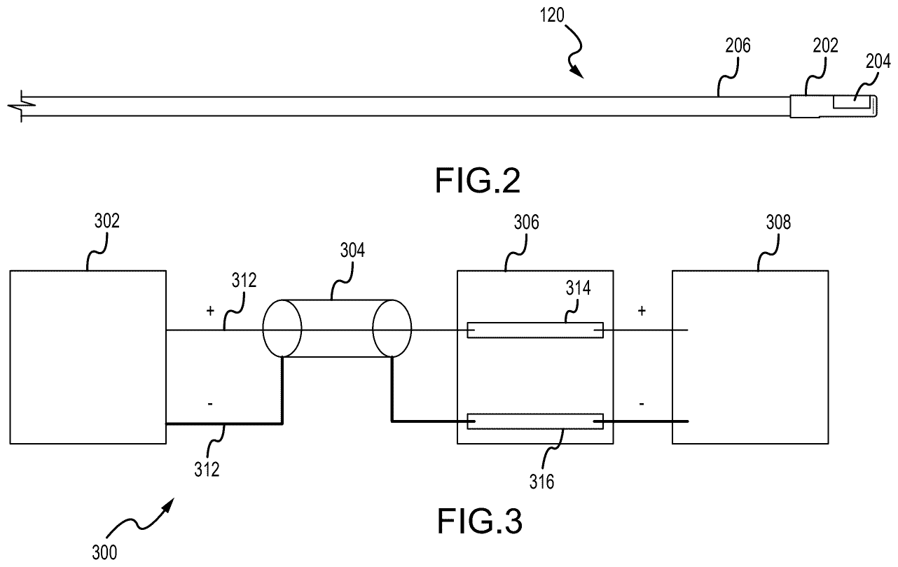

Turning in the drawings, FIG. 2 is a side view of a portion of an imaging catheter 120 of the IVUS system 100 of FIG. 1, according to an embodiment. The imaging catheter 120 may include a shaft 206 (such as a torque coil), a housing 202, and a transducer assembly 204. The imaging catheter 120 may be sized and dimensioned to be inserted into and fit within a vessel. In operation with the system 100 the imaging catheter 120 may be used in percutaneous coronary intervention (“PCI”) procedures, including pre-procedure evaluation and post-procedure optimization. The imaging catheter 120 of the IVUS system 100 may be inserted into a vessel and used to assess plaque nature and characteristics, develop appropriate pretreatment strategies, measure lesion length and reference segment vessel diameter, and select appropriate stent diameter, length, and footing points. Post-PCI, the imaging catheter 120 may be to identify stent expansion and apposition and complications such as stent edge entrapment in order to target and optimize the immediate post-PCI outcomes.

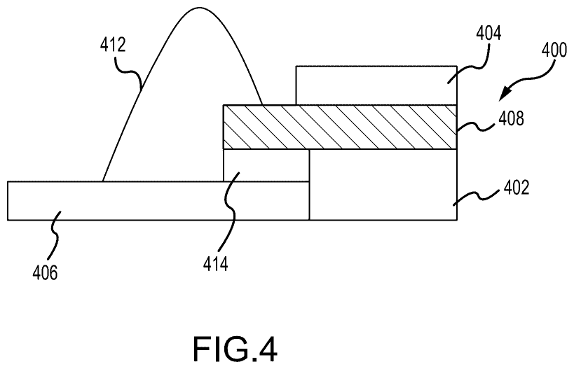

FIG. 3 is a circuit diagram 300 of a transducer assembly 204 of an IVUS system, according to an embodiment. As shown in the circuit diagram 300, the transducer assembly 204 may include a substrate 306 having an impedance matching and bridging circuit 314, 316 (e.g., positive pole 314 and negative pole 316 of a control circuit). In the transducer assembly 204, one or more ultrasound signals are transmitted from a connector 302 of the imaging catheter 120 via wires 312 of a coaxial cable 304 to the substrate 306. In the substrate 306, the impedance matching and bridging circuit 314, 316 is configured to transmit one or more signals to the transducer 308 via a wire bonding 412 (shown in FIG. 4), thus achieving the overall electrical connection from the control system 104 to the transducer 308.

FIG. 4 is a schematic diagram 400 of a transducer assembly 204 of an IVUS system 100, according to an embodiment. The transducer assembly 204 may include a transducer 408 (e.g., transducer 308), a substrate 406 (e.g., substrate 306), a wire 412 electrically connecting the substrate 406 and the transducer 408, a conductive adhesive layer 414, a transducer matching layer 404, and a transducer dissipation layer 402. The transducer 408 may include a piezoelectric element, such as a lead magnesium niobate-lead titanate (PMN-PT) piezoelectric element, inorganic piezoelectric elements (e.g., lead zirconate titanate, zinc oxide, and/or hexagonal boron nitride flakes), organic piezoelectric elements (e.g., polyvinylidene fluoride), composite piezoelectric elements including inorganic (e.g., lead zirconate titanate, zinc oxide, and/or hexagonal boron nitride flakes), organic materials (e.g., polyvinylidene fluoride), bio-inspired materials, or combinations thereof.

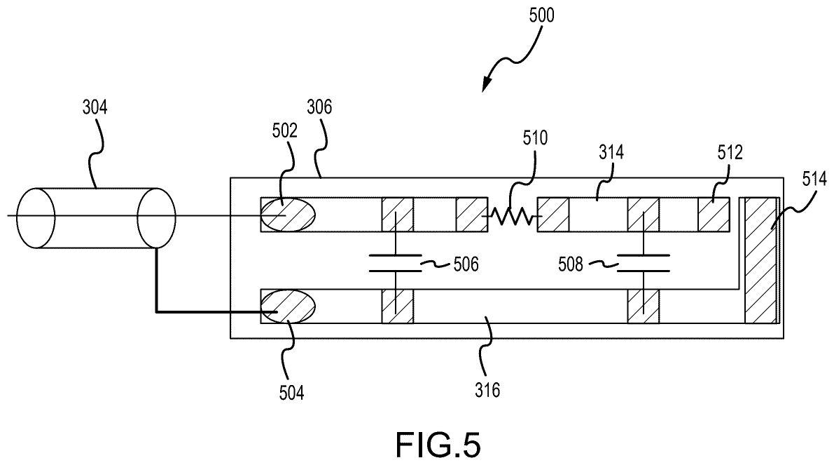

In conventional transducer assemblies, a transducer matching layer, a piezoelectric element, and a transducer dissipation layer are typically stacked together. In the transducer assembly 204 disclosed herein, the substrate 406 is provided and arranged near the transducer dissipation layer 402. The substrate 406 may include a PCB, such as a micro-PCB. The substrate 406 also includes the impedance matching and bridging circuit of FIG. 3 or 5 (e.g., control circuit), which is configured to electrically couple to a cable (e.g., the coaxial cable 304) of the imaging catheter 120 of the IVUS system 100.

The conductive adhesive layer 414 may be positioned between at least a portion of the transducer 408 and at least a portion of the substrate 406. For example, the substrate 406 may be electrically connected to the bottom side of the transducer 408 through the conductive adhesive layer 414, with the conductive adhesive layer 414 electrically therefore connecting the transducer 408 and the substrate 406. In some embodiments, the conductive adhesive layer 414 may be positioned between the bottom side or surface of the transducer and a corresponding pin of the substrate 406. The conductive adhesive layer 414 may include conductive epoxy with silver, gold, other suitable conductive material, or combinations thereof.

The wire 412 electrically connects to the transducer 408 and the substrate 406, with the wire 412 being configured to transmit one or more signals from the impedance matching and bridging circuit (e.g., control circuit) of the substrate 406 to the transducer 408. In some embodiments, the wire 412 may be connected to a top surface of the transducer 408 to a corresponding pin of the substrate 406. The wire 412 may include a bonding wire or semiconductor wire connected to the impedance matching and bridging circuit on the substrate 406 (shown in greater detail in FIG. 5) at a first region of the wire 412 and also connected to the transducer 408 through a wire bonding process at a second region of the wire 412. Wire bonding may be used to create electrical interconnections between a semiconductor device and its packaging during the manufacturing of impedance matching and bridging circuit. In many embodiments, the wire bonding process may include placing a semiconductor die is on the substrate 406, cleaning the bond pads on the die and the substrate to ensure good adhesion, and wire bonding a thin wire (usually gold, aluminum, or copper) to connect the bond pads on the die to the bond pads on the substrate. Wire bonding may utilize one or more of thermosonic bonding, thermocompression bonding, or ultrasonic bonding. The wire bonding process also may include encapsulating the bonded die to protect the bonded die from environmental damage, and testing the final product to ensure it meets the required specifications. Relative to soldering, wire bonding allows for more creation of more fine and precise electrical connections that requires less area on the transducer (e.g., less dead area on the transducer, resulting in better performance of the transducer).

The transducer 408 may be positioned between the transducer matching layer 404 and the transducer dissipation layer 402. The matching layer 404 may include an acoustic matching layer. The matching layer 404 is configured to help transfer ultrasound energy and may include one or more materials conducive to achieve energy transfer, such as epoxy, polyurethan, polystyrene, and/or combinations thereof. The matching layer 404 is configured to help transfer the ultrasound energy from the piezoelectric element 408 to the medium (e.g., the vessel). The matching layer(s) 404 may be located between the piezoelectric element 408 and a lens (not shown). The matching layer 404 may include materials that are conducive to achieving better energy transfer, such as epoxy, polyurethane, polystyrene, and inorganic powders (Ag, Silicon, LiNbO₃, Al₂O₃, etc.) and/or layers (ZnO, etc.). While one matching layer 404 is shown, the matching layer 404 may include multiple (e.g., two or three) matching layers 404. The transducer dissipation layer may include epoxy and tungsten powder to absorb the signals from the non-working side of the transducer assembly.

FIG. 5 is a circuit diagram 500 of substrate 306 (e.g., substrate 406) of a transducer assembly 204 of IVUS system 100, according to an embodiment. The circuit diagram 500 depicts an embodiment of the substrate 306 that includes the impedance matching and bridging circuit 314, 316 (e.g., the impedance matching network design or control circuit). The substrate 500, for example, may include a first capacitor 506, a second capacitor 508, and a resistor 510 positioned between the first capacitor 506 and the second capacitor 508. The first capacitor 506, a second capacitor 508, and the resistor 510 may together constitute or form the impedance matching and bridging circuit. The impedance matching and bridging circuit may be used to achieve impedance matching for different piezoelectric crystals of a transducer 408 by corresponding device parameters of the IVUS system 100 to achieve the best transmission efficiency of electrical energy. In other words, the impedance matching and bridging circuit may allow the transducer assembly 204 to be used effectively with different piezoelectric crystals.

The substrate 306 also includes one or more electrical pads configured to electrically connect components to the substrate 306. For example, the substrate 306 may include electrical pads 502, 504 positioned and configured to electrically connect to one or more (e.g. both) of a mandrel or a shield of the cable 304, respectively. The substrate 306 also may include an electrical pad 512 configured to connect to the wire 412 when wire bonded. The substrate 306 also may include an electrical pad 514 configured to connect to the conductive adhesive layer 414. The one or more electrical pads are electrically connected or configured to electrically connect to the impedance matching and bridging circuit.

Compared with the conventional transducer assemblies, the transducer assembly 204 disclosed herein may include a substrate 406, a wire 412 connecting the substrate 406 and the transducer 408, and an inverted packaging process to achieve the assembling of the substrate 406 and transducer 408. By using the wire bonding process to connect the substrate 406 and the transducer 408 with the wire 412, the solder joints on the transducer can be greatly reduced and/or eliminated compared with the conventional transducer assemblies. The reduction of solder joints improves the effective area of the piezoelectric element of the transducer 408, which directly affects the transmitting and receiving performance of the piezoelectric wafer of the transducer assembly 204. This process of manufacture and resulting configuration can achieve higher production consistency and production efficiency.

The impedance matching and bridging circuit of the substrate also improves the electrical energy transmission efficiency of the transducer assembly 204 in comparison to conventional transducer assemblies. The impedance matching and bridging circuit can achieve better transducer transmitting and receiving performance for the transducer assembly 204 in comparison to conventional transducer assemblies.

Also disclosed herein are imaging catheters and IVUS systems that include the any embodiments of the transducer assemblies described above. For example, the imaging catheter 120 (shown in FIG. 2) may include a shaft 206 having a distal end region, a housing 202 secured to the distal end region of the shaft 206, a cable 304 (shown in FIGS. 3 and 5) extending through the shaft 120 and operably coupled or configured to operably couple to the control system 104 (shown in FIG. 1) of the IVUS system 100, and a transducer assembly 204 secured to the housing 202.

The transducer assembly 204 in the imaging catheter 120 and the IVUS system 100 may include the transducer 308, 408, the substrate 306, 406 electrically coupled to the cable 304, and the wire 412 electrically connected to the transducer 308, 408 and the substrate 306, 406, the wire 412 being configured to transmit one or more signals from the impedance matching and bridging circuit of the substrate 306, 406 to the piezoelectric element of the transducer 308, 408. In order to produce the desired image from the imaging catheter 120, the transducer 308, 408 may receive one or more electrical signals from the control system 104, and the electrical stimulation of the one or more electrical signals causes the piezoelectric element in the transducer 308, 408 to expand and contract to produce high-frequency ultrasound waves. These high-frequency ultrasound waves may be scattered and reflected at the tissue interface, and some of the reflected ultrasound waves are converted back to electrical signals by the transducer 308, 408. These electrical signals from the reflected ultrasound waves may be analyzed and processed by the imaging reconstruction module 110 in the control system 104 for conversion into grayscale cross-sectional images.

The transducer assembly 204 in the imaging catheter 120 and the IVUS system 100 also may include the conductive adhesive layer 414 positioned between at least a portion of the transducer 408 and at least a portion of the substrate 406, with the conductive adhesive layer 414 electrically connecting the transducer 408 and the substrate 406. The transducer assembly 204 in the imaging catheter 120 and the IVUS system 100 also may include the transducer matching layer 404 and the transducer dissipation layer 402, with the transducer 408 being positioned between the transducer matching layer 404 and the transducer dissipation layer 402. In the transducer assembly 204 in the imaging catheter 120 and the IVUS system 100, the substrate 308, 408 may include one or more electrical pads and the cable includes a mandrel and a shield soldered to the one or more electrical pads. In the transducer assembly 204 in the imaging catheter 120 and the IVUS system 100, the impedance matching and bridging circuit of the transducer assembly 308, 408 may include a first capacitor 506, a second capacitor 508, and a resistor 510 positioned between the first capacitor 506 and the second capacitor 508, with the impedance matching and bridging circuit configured to match impedance for the transducer assembly 308, 408 with one or more parameters of the IVUS system 100. In the transducer assembly 204 in the imaging catheter 120 and the IVUS system 100, the substrate 306, 406 may include an electrical pad and the wire 412 is electrically connected to the electrical pad.

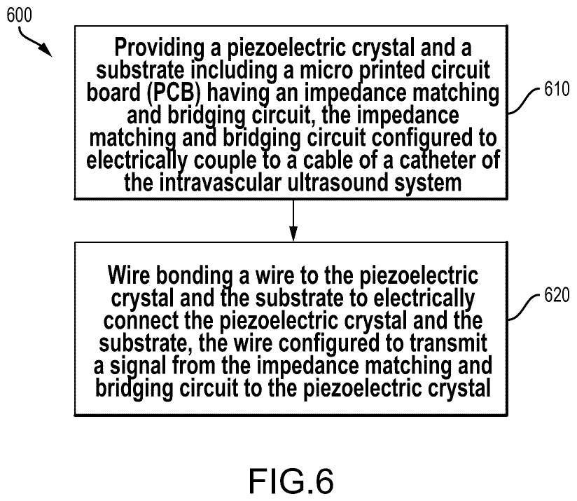

FIG. 6 is a flow diagram of a method 600 of manufacturing a transducer assembly, according to an embodiment. The transducer assembly manufacture according to the method 600 may include any transducer assembly (e.g., embodiments of the transducer assembly 204) disclosed herein. The method 600 includes providing 610 a piezoelectric element (e.g., a transducer) and a substrate including a PCB having an impedance matching and bridging circuit. The impedance matching and bridging circuit is configured to electrically couple to a cable of a catheter of the intravascular ultrasound system. The method 600 also may include wire bonding 620 a wire to the piezoelectric element and the substrate to electrically connect the piezoelectric element and the substrate. The wire is configured to transmit one or more signals from the impedance matching and bridging circuit to the piezoelectric element. Wire bonding may be used to create electrical interconnections between a semiconductor device and its packaging during the manufacturing of impedance matching and bridging circuit. In many embodiments, the wire bonding process may include placing a semiconductor die is on the substrate, cleaning the bond pads on the die and the substrate to ensure good adhesion, and wire bonding a thin wire (usually gold, aluminum, or copper) to connect the bond pads on the die to the bond pads on the substrate. Wire bonding may utilize one or more of thermosonic bonding, thermocompression bonding, and/or ultrasonic bonding. The wire bonding process also may include encapsulating the bonded die to protect the bonded die from environmental damage, and testing the final product to ensure it meets the required specifications. Relative to soldering, wire bonding allows for more creation of more fine and precise electrical connections that requires less area on the transducer (e.g., less dead area on the transducer, resulting in better performance of the transducer).

The method 600 also may include positioning a conductive adhesive layer between at least a portion of the piezoelectric element and at least a portion of the substrate, with the conductive adhesive layer electrically connecting the piezoelectric element and the substrate. The method 600 also may include positioning the piezoelectric element between a transducer matching layer and a transducer dissipation layer. The method 600 also may comprise positioning a first capacitor, a second capacitor, and a resistor on the impedance matching and bridging circuit with the resistor positioned between the first capacitor and the second capacitor. The impedance matching and bridging circuit including the first capacitor, the second capacitor, and the resistor may be configured to match impedance for the piezoelectric element with one or more parameters of the IVUS system.

The method 600 also may include positioning one or more electrical pads on the substrate, with the one or more (e.g., two) electrical pads being configured to electrically connect to the one or more (e.g., both) of a mandrel or a shield of the cable. The method also may include electrically connecting the one or more (e.g., both) of the mandrel and the shield of the cable to the one or more (e.g., two) electrical pads. The method 600 also may comprise positioning an electrical pad on the substrate to electrically connect the electrical pad on the substrate to the wire.

Acts of the method 600 are for illustrative purposes. For example, acts of the method 600 may be performed in different orders, split into multiple acts, modified, supplemented, or combined.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

What is claimed is:

1. A transducer assembly for an intravascular ultrasound system, the transducer assembly comprising:
a piezoelectric element;
a substrate including a printed circuit board (“PCB”) having a control circuit, the control circuit configured to electrically couple to a cable of a catheter of the intravascular ultrasound system; and
a wire electrically connected to the piezoelectric element and the substrate, the wire configured to transmit one or more signals between the control circuit and the piezoelectric element.

2. The transducer assembly of claim 1, further comprising a conductive adhesive layer positioned between at least a portion of the piezoelectric element and at least a portion of the substrate, the conductive adhesive layer electrically connecting the piezoelectric element and the substrate.

3. The transducer assembly of claim 1, further comprising a transducer matching layer and a transducer dissipation layer, the piezoelectric element being positioned between the transducer matching layer and the transducer dissipation layer.

4. The transducer assembly of claim 1, wherein the substrate includes one or more electrical pads positioned and configured to electrically connect to one or more of a mandrel or a shield of the cable.

5. The transducer assembly of claim 1, wherein the control includes a first capacitor, a second capacitor, and a resistor positioned between the first capacitor and the second capacitor.

6. The transducer assembly of claim 5, wherein the control circuit including is configured to match impedance for the piezoelectric element with one or more parameters of the intravascular ultrasound system.

7. The transducer assembly of claim 1, wherein the substrate includes an electrical pad positioned and configured to electrically connect to the wire.

8. An imaging catheter for an intravascular ultrasound system, the imaging catheter comprising:
a shaft having a distal end region;
a housing secured to the distal end region of the shaft;
a cable extending through the shaft and configured to operably couple to a control system of the intravascular ultrasound system; and
a transducer assembly secured to the housing, the transducer assembly including:
a piezoelectric element;
a substrate including a printed circuit board (“PCB”) having a control circuit electrically coupled to the cable; and
a wire electrically connected to the piezoelectric element and the substrate, the wire configured to transmit one or more signals between the control circuit and the piezoelectric element.

9. The imaging catheter of claim 8, further comprising a conductive adhesive layer positioned between at least a portion of the piezoelectric element and at least a portion of the substrate, the conductive adhesive layer electrically connecting the piezoelectric element and the substrate.

10. The imaging catheter of claim 8, wherein the transducer assembly includes a transducer matching layer and a transducer dissipation layer, the piezoelectric element being positioned between the transducer matching layer and the transducer dissipation layer.

11. The imaging catheter of claim 8, wherein the substrate includes one or more electrical pads and the cable includes a mandrel or a shield soldered to the one or more electrical pads.

12. The imaging catheter of claim 8, wherein the control circuit of the transducer assembly includes a first capacitor, a second capacitor, and a resistor positioned between the first capacitor and the second capacitor.

13. The imaging catheter of claim 12, wherein the control circuit including the first capacitor, the second capacitor, and the resistor is configured to match impedance for the piezoelectric element with one or more parameters of the intravascular ultrasound system.

14. The imaging catheter of claim 8, wherein the substrate includes an electrical pad and the wire is electrically connected to the electrical pad.

15. An intravascular ultrasound system, comprising:
a control system; and
an imaging catheter operably coupled to the control system, the imaging catheter comprising a shaft having a distal end region, a housing secured to the distal end region of the shaft, a cable extending through the shaft and operably coupled to the control system, and a transducer assembly secured to the housing, the transducer assembly including:
a piezoelectric element;
a substrate including a micro printed circuit board (PCB) having a control circuit electrically coupled to the cable; and
a wire electrically connected to the piezoelectric element and the substrate, the wire configured to transmit one or more signals between the control circuit and the piezoelectric element.

16. The intravascular ultrasound system of claim 15, wherein the imaging catheter includes a conductive adhesive layer positioned between at least a portion of the piezoelectric element and at least a portion of the substrate, the conductive adhesive layer electrically connecting the piezoelectric element and the substrate.

17. The intravascular ultrasound system of claim 15, wherein the transducer assembly includes a transducer matching layer and a transducer dissipation layer, the piezoelectric element being positioned between the transducer matching layer and the transducer dissipation layer.

18. The intravascular ultrasound system of claim 15, wherein the substrate includes one or more electrical pads and the cable includes a mandrel or a shield soldered to the one or more electrical pads.

19. The intravascular ultrasound system of claim 15, wherein the control circuit of the transducer assembly includes a first capacitor, a second capacitor, and a resistor positioned between the first capacitor and the second capacitor.

20. The intravascular ultrasound system of claim 19, wherein the control circuit including the first capacitor, the second capacitor, and the resistor is configured to match impedance for the piezoelectric element with one or more parameters of the intravascular ultrasound system.

21. The intravascular ultrasound system of claim 15, wherein the substrate includes an electrical pad and the wire is electrically connected to the electrical pad.

22. A method of assembling a transducer assembly, the method comprising:
providing a piezoelectric element and a substrate including a micro printed circuit board (PCB) having a control circuit, the control circuit configured to electrically couple to a cable of a catheter of the intravascular ultrasound system; and
wire bonding a wire to the piezoelectric element and the substrate to electrically connect the piezoelectric element and the substrate, the wire configured to transmit one or more signals between the control circuit and the piezoelectric element.

23. The method of claim 22, further comprising positioning a conductive adhesive layer between at least a portion of the piezoelectric element and at least a portion of the substrate, the conductive adhesive layer electrically connecting the piezoelectric element and the substrate.

24. The method of claim 22, further comprising positioning the piezoelectric element between a transducer matching layer and a transducer dissipation layer.

25. The method of claim 22, further comprising positioning one or more electrical pads on the substrate, the one or more electrical pads being configured to electrically connect to one or more of a mandrel or a shield of the cable.

26. The method of claim 22, further comprising positioning a first capacitor, a second capacitor, and a resistor on the control circuit with the resistor positioned between the first capacitor and the second capacitor.

27. The method of claim 26, wherein the control circuit including the first capacitor, the second capacitor, and the resistor is configured to match impedance for the piezoelectric element with one or more parameters of the intravascular ultrasound system.

28. The method of claim 22, further comprising positioning an electrical pad on the substrate to electrically connect the electrical pad on the substrate to the wire.
